(19) United States
(12) Patent Application Publication
(10) Pub. No.: US 2021/0401736 A1
(43) Pub. Date: Dec. 30, 2021

(54) SEMI-SOLID CHEWABLE GEL CANNABINOID COMPOSITIONS AND METHODS OF MAKING AND USING THEREOF

(71) Applicant: Seattle Gummy Company, Seattle, WA (US)

(72) Inventors: Feng Wan, Sammamish, WA (US); William Brenden Carlson, Shoreline, WA (US)

(73) Assignee: Seattle Gummy Company, Seattle, WA (US)

(21) Appl. No.: 15/931,604

(22) Filed: May 14, 2020

Related U.S. Application Data
(60) Provisional application No. 62/847,946, filed on May 15, 2019.

(51) Int. Cl. A61K 9/00; A61K 31/05; A61K 31/352; A61K 47/40; A61K 47/26; A61K 45/06

(52) U.S. Cl. CPC A61K 9/0056; A61K 31/05; A61K 45/06; A61K 47/40; A61K 47/26; A61K 31/352

(57) ABSTRACT

A semi-solid chewable gel composition, comprising a gelling component in a sufficient amount to provide a cohesive gelled product, a cannabinoid component comprising cannabidiol, and a complexing component, wherein the complexing component is configured to reduce the bitterness of the cannabinoid component by complexing with the cannabinoid component.

### CROSS-REFERENCES TO RELATED APPLICATIONS

This application claims the benefit of priority from, and hereby incorporates by reference the entire disclosure, co-pending US Provisional Application for Patent Ser. No. 62/847,946, filed May 15, 2019.

### TECHNICAL FIELD

The application relates generally to nutraceutical composition in semi-solid chewable gel or gummy formula, methods of administration of various gummy or gelled compositions for bioactive compounds, and kits comprising various gummy composition.

### BACKGROUND

The cannabis plant has many naturally occurring substances including terpenes and cannabinoids that are of various biological activities. Isolated alkaloid compounds from the cannabis plant are called cannabinoids. There are over one hundred and forty cannabinoids that have been isolated from the cannabis plant including delta9-tetrahydrocannabinol (THC), cannabidiol (CBD), cannabichromene (CBC), cannabigerol (CBG), cannabinol (CBN), cannabidivarin (CBDV). While THC has psychoactive effects, CBD, CBC, CBG, and CBDV do not.

Cannabinoids can be isolated by extraction or cold pressing from cannabis plants. Plants in the cannabis genus include Cannabis sativa, Cannabis ruderalis, and Cannabis indica. These plants are natural sources of cannabinoids. Cannabinoids are also available in synthetic forms.

Cannabidiol is a major phytocannabinoid, accounting for up to 40% of the plant's extract. CBD is a CB-1 receptor antagonist, while THC is a CB-1 receptor agonist. Research has found that cannabis strains with higher concentration of CBD did not produce the short-term memory impairment normally seen in high THC cannabis strain, a characteristic attributed to the CB-1 receptor antagonist nature of CBD. CBD is considered to have a wider scope of medical applications than THC.

Cannabigerol (CBG) is a non-psychoactive cannabinoid found in the cannabis plant. All cannabinoids in the early stage of the cannabis plant's life begin as CBG. CBG is found in higher concentrations in hemp plants as opposed to marijuana plants, which are grown to have higher concentrations of tetrahydrocannabinol (THC). CBG has been found to act as a high affinity alpha2-adrenergic receptor agonist, a moderate affinity to 5-H1A receptor antagonist, and a low affinity CB1 receptor antagonist. It binds with the CB2 receptor.

Many researches have confirmed the medicinal value of cannabinoids. Cannabinoids have been investigated for possible treatment of seizures, nausea, vomiting, lack of appetite, pain, arthritis, inflammation, neurological disorders, and other conditions.

CBD, CBN, CBG and the other cannabidiol derivatives have many medicinal uses. A double-blinded, randomized, placebo-controlled study of CBD oil on treating Dravet syndrome, a rare form of epilepsy that begins in infancy and is difficult to treat, was conducted in 2017 and found that CBD oil significantly reduced the number of seizures with a dosing of 20 mg/kg. CBN acts as a partial agonist at the CB₁ receptors but has a higher affinity to CB₂ receptors; as such, CBN promotes pain relief and is an anti-inflammatory. CBN's most pronounced, characterizing attribute is its sedative effect, and according to studies, 5 mg of CBN is as effective as 10 mg dose of diazepam, a mild pharmaceutical sedative. CBG has been found to act as a high affinity a2-adrenergic receptor agonist, moderate affinity 5-HT1A receptor antagonist, and low affinity CB₁ receptor antagonist. CBG also binds to the CB₂ receptor as an antagonist.

Drugs with cannabidiol derivatives as active pharmaceutical ingredient (API) currently on the market are Epidiolex by GW Pharmaceuticals for the treatment of Dravent, Marinol by AbbVie for treatment of AIDS and cancer patients that have lost appetite, and Sativex by Bayer to alleviate neuropathic pain and spasticity.

### SUMMARY

A semi-solid chewable gel (i.e., gummy) is a chewable formulation that comprises a gelling component, a binding component, and water. The gelling component may comprise gelling agents such as pectin, starch and gelatin. The binding component may include binding agents. Example binding agent comprises simple mono and disaccharides (or carbohydrates), sugar alcohols, etc. Simple monosaccharides include glucose, fructose, and disaccharides include sucrose. Other carbohydrates include sugar alcohols such as sorbitol, and higher oligomer carbohydrates such as maltodextrin. Sometimes a flavor and/or color is used with the gelling agents and sugars. The formulation may have a water content of less than 25%, 20%, 15%, 12% or 10%.

In one aspect, the application provides semi-solid chewable gel (gummy) formulations for delivery of cannabinoids actives. Gummy products for CBD, CBN, CBG, and other cannabidiol derivatives delivery have been designed, formulated and characterized. The formulation disclosed herein provides advantages include, without limitation, that: 1) gummy taste good with significant flavor masking or removal of bitterness from cannabinoids actives; as such, delivery can take place with pleasing sensory perception; and 2) gummy formulation dissolves and coats the mucosal membrane, allowing for fast absorption of the actives through a trans-mucosal process and bypassing the digestion track.

In one embodiment, the gummy composition with cannabidiol derivatives has excellent taste, chew, appearance, and texture. The gummy has a number of advantages including allowing the hydrophobic cannabinoids to be fully dissolved or uniformly dispersed in the hydrophilic gummy matrix. In one embodiment, the complexing coordination or inclusion complex formation between the complexing component and cannabinoid component significantly increased the solubility, dispersibility, flavor and/or bioavailability of cannabidiols in the gummy matrix.

In one embodiment, the gummy consists of either a gelatin or pectin as gelling agent and sucrose, fructose, citric acid, colorant, and flavor. Because cannabinoids have the bitter taste, extensive research and development was conducted by the applicant to alleviate the bitter taste of cannabinoids in the disclosed gummy formulations. In one embodiment, the gummy formulation comprises one or more complexing agent that coordinates the cannabidiol derivatives molecularly. In one embodiment, the complexing agent comprises a cyclic carbohydrate molecule, such as a cyclic sugar, having a molecule made of glucose units arranged in ring or crown structure. In one embodiment, the complexing agent complexes cannabinoids molecule by coordinating therefore holding at least part of the cannabinoid molecule inside the glucose ring. The complexing effect of the glucose ring allows for the hydrophobic cannabinoid molecules to be dissolved (be soluble) into the hydrophilic gummy matrix. The complexing effect also allows for a better flavor and texture of the disclosed gummy composition by reducing or eliminating the bitter cannabinoid taste.

In one embodiment, the semi-solid chewable gel composition comprises a gelling component in a sufficient amount to provide a cohesive gelled product, a cannabinoid component comprising cannabidiol, and a complexing component, wherein the complexing component is configured to reduce the bitterness of the cannabinoid component by complexing with the cannabinoid component.

The cannabinoid component may include cannabidiol (CBD), delta9-tetrahydrocannabinol (THC), cannabichromene (CBC), cannabigerol (CBG), cannabinol (CBN), cannabidivarin (CBDV), a cannabis plant extract or isolate, a hemp extract or isolates, a marijuana extract or isolate, or a combination thereof. In one embodiment, the cannabinoid component comprises a cannabis plant extract or powder containing at least 1%, 2%, 5%, 10%, 20%, 30%, 40%, 50%, 55%, 60%, 70%, 80%, 90%, 98%, 99% of cannabidiol. In one embodiment, the cannabinoid component comprises THC. In one embodiment, the cannabinoid component comprises substantially cannabidiol.

In one embodiment, the cannabinoid component comprises a cannabidiol isolate. In one embodiment, the cannabidiol isolate is an oil or a crystalline solid. In one embodiment, the cannabidiol isolate comprises from about 1% to about 99% cannabidiol. The cannabidiol isolate may be derived from a marijuana plant or a hemp plant. In one embodiment, the cannabinoid isolate comprises less than 0.3% of THC.

In one embodiment, the cannabinoid component comprises synthetic cannabidiol. In one embodiment, the semi-solid chewable composition may include cacao derivatives. The cocoa derivative may include theobromine, polyphenol, flavonoids, or a combination thereof.

The complexing component is configured to complex with cannabidiol thereby reducing the bitterness of cannabidiol, increasing the solubility or dispersibility of cannabidiol derivatives into the gummy matrix. The complexing component may include nucleic acid, nucleotide, nucleic acid base, fruit powder, protein, peptide, cluster dextrin, cyclodextrin, polydextrose, polyethylene glycol, fatty acids, waxes, zeolite, chitosan, poly N-acetyglucosamine, N-acetylglucosamine, or a combination thereof.

In one embodiment, the semi-solid composition comprises at least 0.5% the complexing component by weight. In one embodiment, the semi-solid composition comprises at least 3% the complexing component by weight. In one embodiment, the complexing component comprises cyclodextrin and wherein the semi-solid composition comprises at least 0.1% of cyclodextrin.

In one embodiment, the complexing component comprises a plant powder. In one embodiment, the plant powder is a nucleic acid rich fruit powder including, for example, strawberry powder, cantaloupe powder, raspberry powder, blueberry powder, acai berry powder, goji berry powder, jujube powder, dates powder, melon powder, or a combination thereof. In one embodiment, the semi-solid composition comprises at least 0.1%, 0.2%, 0.5%, 0.8%, or 1% of the fruit powder.

The gelling component may include gelatin, starch, pectin, gellan gum, gum Arabic, carrageenans, guar, agar, alginate, locust bean gum, xanthan, or a combination or derivatives thereof. In one embodiment, the gelling component consists essentially of pectin having a methoxy content of not less than 30%, 35%, 40%, 42%, 45%, 46%, 47%, or 50%. In one embodiment, the gelling component consists essentially of pectin having an amid content of not less than 10%, 12%, 15%, 18%, 20%, or 21%.

The semi-solid chewable gel composition may further include a binding component. In one embodiment, the binding component interacts or cross-links with gelling agent to form the semi-solid gel composition. In one embodiment, the binding component comprises sucrose, glucose, fructose, palatinose, trehalose, psicose, tagatose, sorbose, a sugar alcohol, maltodextrin, resistant maltodextrin, glycerol, or a combination thereof. In one embodiment, the binding component comprises essentially sucrose, glucose, fructose, or a combination thereof. In one embodiment, the binding component comprises essentially trehalose, palatinose, psicose, tagatose, sorbose, or a combination thereof. In one embodiment, the binding component comprises essentially trehalose, palatinose, psicose, resistant maltodextrine, or a combination thereof. In one embodiment, the binding component comprises essentially trehalose, palatinose, psicose, tagatose, maltodextrine, or a combination thereof.

In one embodiment, the binding component comprises essentially sugar alcohols. In one embodiment, the binding component comprises sorbitol, xylitol, maltitol, isomalt, lactitol, mannitol, erythritol, hydrogenated starch hydrolysates, or a combination thereof.

In one embodiment, the semi-solid chewable gel composition comprises at least 0.2%, 0.5%, 1%, 1.5%, 2%, 2.5%, or 3% cannabidiol by weight. In one embodiment, the semi-solid chewable gel composition comprises from about 0.01% to about 2% cannabidiol by weight.

The semi-solid chewable gel composition may further include an antioxidant composition, a vitamin composition, a mineral composition, an amino acid composition, an herb composition, a prebiotic composition, a probiotic composition, an active composition, or a combination thereof.

In one embodiment, the antioxidant composition comprises vitamin A, vitamin E, vitamin C, beta-carotene, alpha-carotene, lycopene, lutein, folic acid, gallic acid, resveratrol, quinone, Coenzyme Q10, selenium, selenium yeast, phenolics, polyphenols, anthocyanins, flavonoids, gingko biloba, astaxanthin, canthaxanthin, cryptoxanthin, anthracenes, carotenoids, zeaxanthin, curcumin, or derivatives thereof.

In one embodiment, the vitamin composition comprises vitamin A, B, C, D, E, K or a combination thereof.

In one embodiment, the mineral composition comprises salts of calcium, iron, zinc, magnesium, sodium, chloride, potassium, copper, molybdenum, manganese, phosphorus, iodine, nickel, or selenium, or a combination thereof.

In one embodiment, the amino acid composition comprises an essential amino acid, a branch-chain amino acid, a stimulant amino acid, glycine, tryptophan, L-theanine, S-adenosyl methionine, 5-hydroxytryptophan, or its derivative thereof.

The herbal component may work synergistically with cannabinoids to enhance bioactivity, reduce side effects or both. In one embodiment, the herbal component comprises gingko biloba, turmeric, ginger, astragalus, Prunella vulgaris, Pueraria montana var. lobata, Salvia miltiorrhiza, Coptis chinensis, Eucommia ulmoides Oliver, cranberry, blackberry, elderberry extract, cranberry, blueberry, grapeseed, saffron, Sangre de grado (dragon's blood), green tea, ginseng, Ashwagandha, chamomile, lavender, passion flower, Magnolia bark, Valerian root, lemon balm, kava, St. John's wort, Rhodiola (Rhodiola Rosea), maca, rosemary, thyme, peony, Albizia julibrissin, icariin, Epimedium, Perilla frutescens, various mushrooms or fungi, its extract, powder or derivative thereof. In one embodiment, the herbal component comprises essentially gingko biloba. Not wanting to be limited by theory, gingko biloba, an herbal active known to promote cerebral blood flow has been observed through applicant's research to enhance the efficacy or bioactivity of cannabinoids when combined in the disclosed formulation potentially by increasing the reach of cannabinoids to the brain region.

In one embodiment, the active composition comprises glucosamine, chondroitin, melatonin, gamma-aminobutyric acid, or a combination thereof.

The semi-solid composition may further include an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the sweetener comprises erythritol, xylitol, sucrose, fructose, glucose, maltose, juice or juice concentrate, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylalanine 1-methyl ester, salts thereof, licorice or its extracts or isolates, stevia or its extracts or isolates, monk fruit or its extracts or isolates, or a mixture thereof.

The semi-solid chewable gel composition may have a glycemic index of not more than 25. In one embodiment, the semi-solid chewable gel composition has a glycemic index of not more than 20, 15, 10, 8, or 5. In one embodiment, the semi-solid chewable gel composition has a glycemic index of less than 1. In one embodiment, the semi-solid chewable gel composition has a glycemic index of 0.

The semi-solid chewable gel composition may have a high glycemic index of more than 60, 70, 80 or 90. In one embodiment, the composition may have a glycemic index of more than 75. In one embodiment, the composition may have a glycemic index of more than 65.

In one embodiment, the semi-solid chewable gel composition is substantially sugar free. In one embodiment, the semi-solid chewable gel composition is substantially free of sucrose, glucose, and fructose, sugar alcohol or a combination thereof.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the chemical structure of example cannabinoids, (left) cannabidiol (CBD), (middle) Cannabinol (CBN), and (right) Cannabigerol (CBG);

FIG. 2 shows that trehalose digests and releases glucose at a much slower rate when compared to other sugars;

FIG. 3 shows that, similar to trehalose, palatinose digests slowly and has much lower impact on blood glucose levels than other sugars;

FIG. 4 shows the simplified structure of cluster dextrin, in which cluster dextrin is shown to have a ring structure with pendent chains of glucose with a tendency of forming helical structures making it suitable for complexing with cannabidiol derivatives;

FIG. 5 shows the ring structures of alpha, beta, and gamma cyclodextrin, wherein alpha cyclodextrin consists of a ring of 6 glucose units linked through 1,4-beta-glycosidic bonds, beta cyclodextrin consists a ring of seven glucose units and gamma comprises a ring of eight glucose units; and

FIG. 6 shows the ring structure of the cyclodextrin coordination of (complexign with) the CBD molecule, wherein the interior of the cylcodextrin is able to electronically interact with the phenyl groups of the CBD molecule, the phenyl group is a reverse quadrapole where the interior of the aromatic ring contains high electron density and the exterior of the ring is electron deficient, the hydrogen atoms of the hydroxyl groups of the cyclodextrin are electronically attracted to the pi system of the aromatic ring, and the hydrogen atoms of the aromatic ring are electronically attracted to the oxygen atoms of the cyclodextrin.

### DETAILED DESCRIPTION

The present disclosure is capable of being embodied in various forms. The description below of several embodiments is made with the understanding that the present disclosure is to be considered as an exemplification of the claimed subject matter and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in its non-limiting sense to mean those items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more.” The word “cannabinoid” used in this description, claims, and other conjugations is used to mean any compound that interacts with a cannabinoid receptor and other cannabinoid mimetics, including, but not limited to, certain tetrahydropyran analogs (delta9-tetrahydrocannabinol, delta8-tetrahydrocannabinol, 6,6,9-trimythel-3-pentyl-6H-dibenzo[b,d]pyran-1-ol, 3-(1,1-dimethylheptyl)--6,6a7,8,10,10a-hexahydro-1-1hydroxy-6,6-dimethyl-9H-dibezo[b,d]pyran-9-ol-, (−)-(3S,4S)-7-hydroxy-delta-6-tetrahydrocannabinol-1,1-dimethylheptyl, (+)-(3S,4S)-7-hydroxy-delta-6-tetrahydrocannabinol, and delta8-tetrahydrocannabinol-11-oic acid); certain piperidine analogs (e.g., (−)-(6S,6aR,9R,10aR)-5,6,6a,7,8,9,10,10a-octahydro-6-methyl-1-3-[(-R)-1-methyl-4-phenylbutoxy]-1,9-phenanthridinediol 1-acetate)); certain aminoalkylindole analogs (e.g., (R)-(+)-[2,3-dihydro-5-methyl-3-(4-morpholinylm-ethyl)-pyrrolo[1,2,3,-de]- -1,4-benzoxazin-6-yl]-1-naphthelenyl-methanone); certain open pyran-ring analogs (e.g., 2-[3-methyl-6-(1-methylethenyl-2-cyclohexen-1-yl]-5-pentyl-1,3-benzendi-o-1, and 4-(1,1-dimethylheptyl)-2,3′-dihydroxy-6′-alpha-(3-hydroxypropyl)-1′,-2′,3′,4′,5′,6′-hexahydrobiphenyl), their salts, solvates, metabolites, and metabolic precursors.

The word “cannabidiol” refers to cannabidiol and cannabidiol derivatives. As used in this application, cannabidiol is obtained from industrial hemp extract with a low amount of THC or from cannabis extract using high-CBD cannabis cultivars. Cannabidiol may also be synthetic.

The word “cannabigerol” refers to cannabigerol and cannabigerol derivatives. As used in this application, cannabigerol is derived from industrial hemp extract with a trace amount of THC or from cannabis extract. Cannabigerol may also be synthetic.

Any embodiments with recited numbers or numeric range include any integra or number in between.

A cannabis semi-solid chewable gel (gummy) product that utilizes CBD, CBN, CBG and other cannabidiol derivatives as the active ingredient have been developed. The gummy product is excellent in texture, taste, and flavor and allows for rapid delivery of the cannabidiol derivative active ingredient potentially for fast relief of a number of symptoms and ailments. This form of cannabidiol derivative delivery greatly advances the start of the art of medication delivery and medical application of the cannabidiol derivatives.

Cannabidiol Derivatives

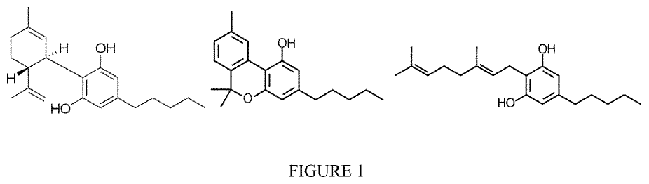

Example cannabidiol derivatives include, among others, CBD, CBN, and CBG, as shown in FIG. 1. The compounds are largely hydrophobic and water insoluble. They have low solubility in polyols such as glycerol. Gummy formulation is based upon a hydrophilic matrix. Therefore, the hydrophobic cannabinoids tend to separate from the hydrophilic gummy mixture.

CBD, CBN, CBG and the other cannabidiol derivatives are non-psychoactive and non-intoxicating compounds. The cannabinoids have numerous medicinal applications. Some medicinal uses for the cannabinoids where they have been shown to be effective are shown in TABLE 1.

TABLE 1

| Cannabinoid | Medicinal Properties and Applications |
|---|---|
| CBD | Epilepsy, Seizures, Multiple Sclerosis, |
| | Relaxation |
| CBN | Anti-inflammatory, Sedative, Pain Relief |
| CBG | Pain relief, anxiety, inflammatory bowel |
| | disease, ulcerative colitis and Crohn's |
| | disease, Glaucoma, Cancer |
| CBC | Anti-viral, Antifungal, w/CBD Anti-depressant |
| THCV | Obesity-associated glucose intolerance |
| CBDV | Anti-epileptic and Anti-convulsive |
| CBDA | Anti-proliferative effect on cancer cells |

Gummy Matrix

Gelling Agents

The texture of gummy compositions mostly comes from the gelling agents. In one embodiment, the gelling agents include high molecular weight polymers. In one embodiment, the gelling agents are complex carbohydrates. In one embodiment, the gelling agents may be proteins.

The gelling component serves to form the polymeric matric providing the semi-solid texture to the semi-solid composition. The semi-solid composition may contain at least 0.5% by weight of the gelling component. In one embodiment, the semi-solid composition contains from about 1.25% by weight to about 8.5% by weight of the gelling component. In one embodiment, the semi-solid component comprises about 0.5%, 0.75%, 1.25%, 1.75%, 2.25%, 2.75%, 3.25%, 3.75%, 4.25%, 4.75%, 5.25%, 5.75%, 6.25%, 6.75%, 7.25%, 7.75%, 8.25%, or 8.75% by weight of the gelling component.

In one embodiment, the gelling component comprises gelatin, donkey hide gelatin, starch, pectin, gellan gum, gum Arabic, carrageenans, guar, agar, alginate, locust bean gum, xanthan, or derivatives thereof. In one embodiment, the gelling component comprises pectin and gelatin in a ratio from about 10:1 to about 1:1. In one embodiment, the gelling component comprises gelatin and starch in a ratio from about 100:1 to about 1:100. In one embodiment, the gelling component comprises gelatin and alginate. In one embodiment, the gelling component comprises alginate and starch. In one embodiment, the gelling component consists essentially of starch, gelatin, alginate or pectin.

In one embodiment, the gelling component consists essentially of pectin. In one embodiment, the gelling component comprises apple pectin, citrus pectin, or a combination thereof. In one embodiment, the semi-solid composition comprises at least 1% of pectin. In one embodiment, the semi-solid composition comprises from about 1% to about 5% of pectin. In one embodiment, the semi-solid composition comprises about 2.5% pectin. In one embodiment, pectin has a methoxy content of not less than 30%, 40% or 50%. In one embodiment, pectin has an amid content of not less than 10%, 15%, 20%, 25%, 30%, 40%, or 45%. In one embodiment, pectin has a carboxylic content of not less than 25%, 30%, 35%, 40%, 50%, or 60%. In one embodiment, the pectin has a methyl ester not more than 30%, 32%, 35%, or 40%.

In one embodiment, the gelling component consists essentially of gelatin or collagen. In one embodiment, the gelling component consists essentially of starch. In one embodiment, the gelling component consists essentially of alginate.

Pectin is a heteropolysaccharide consisting of galactogluconic acid found in plant cell walls that gives plants their rigid structure. Pectin may be the type that gels in the presence of sugar and acid. In another embodiment, pectin may be chemically modified that gels in the presence of calcium or potassium ions. Pectin is water soluble, but it's easier to dissolve in water when mixed with sugar. In one embodiment, pectin may be used in combination with a second gelling agent, such as gelatin. Gelatin provides a tough chewiness, while pectin lends the composition a distinct tenderness.

Gelatin is a protein made from animal collagen. Collagen may be derived from cattle, pigs, and fish. In one embodiment, gelatin is combined with other hydrocolloids (as gelling agents) including pectin, agar, starch, gum Arabic to create unique textures. For example, a combination of gelatin and gum arabic gives the soft chewy texture.

There are two kinds of starch molecules: amylose and amylopectin, both are based upon the alpha form of glucose. Amylose starches gel when heated; amylopectin starches don't. In one embodiment, the disclosed composition comprises modified starch as the gelling agent. There are a variety of starch modification techniques, but common ones include contacting starch with acid, sodium or potassium hydroxide, or oxidizing the starch. These treatments help the starch to dissolve in water and gel to an appropriate level.

Agar or agar-agar is a jelly-like substance, obtained from algae. Agar is derived from the polysaccharide agarose. Agar comprises two components: the linear polysaccharide agarose, and a heterogeneous mixture of smaller molecules called agaropectin. Agar gels tend to weep or extrude water over time when used by itself as a gelling agent. In one embodiment, a combination of agar and locust bean gum are used as gelling agents. Locust bean gum helps to prevent weeping of agar gels. Locust bean gum is a galactomannan polysaccharide vegetable gum extracted from the seeds of the carob tree. The two polysaccharides from agar and locust bean gum synergistically interact with each other to form a strong gel that does not weep.

Carrageenans or carrageenins are a family of linear sulfated polysaccharides that are extracted from red edible seaweeds. The linear saccharide chains have a tendency to curl to form helical structures. Kappa-carrageenan has one sulphate group per disaccharide and forms strong, rigid gels in the presence of potassium ions. In one embodiment, locust bean gum is used with kappa-carrageenan as gelling agents to prevent water from being expelled from the bulk of the gel (weeping). Gels formed from kappa-carrageenan and potassium ions are thermally reversible.

Alginic acid is a linear copolymer with homopolymeric blocks of (1-4)-linked β-D-mannuronate (M) (acid form of mannose) and its C-5 epimer α-L-guluronate (G) (acid form of gulose) residues, respectively, covalently linked together in different sequences or blocks. The monomers can appear in homopolymeric blocks of consecutive G-residues (G-blocks), consecutive M-residues (M-blocks) or alternating M and G-residues (MG-blocks). Alginate forms strong hydrogels when crosslinked with calcium ions.

Simple Carbohydrates

Binding component functions to bind or crosslink gelling agents to form the semi-solid gel composition. The binding component may comprise carbohydrates such as sugars or sugar alcohols. In one embodiment, the carbohydrates help to bind the gummy together through interaction with the gelling agent. In one embodiment, the carbohydrates keep the texture of the composition soft by acting as a humectant. With being limited by theory, the carbohydrates bind water. By binding water, the carbohydrates prevent the semi-solid chewable gel composition from crystalizing, from drying out, and giving the product a chewy texture.

Sucrose, commonly known as table sugar, is a disaccharide consisting of one glucose unit and one fructose unit. The IUPAC name is O-α-D-glucopyranosyl-(1→2)-β-D-fructofuranoside. Sucrose is soluble in water. The interaction between sucrose and water allows for sucrose to serve as a humectant for the composition. In one embodiment, sucrose helps the gummy to retain water and maintain its texture. In one embodiment, sucrose also provides sweetness. In one embodiment, the chewable gel composition comprises invert sugar.

Fructose is a monosaccharide, a ketose. Fructose is water soluble and may be used as a humectant in gummy compositions. Fructose helps the gummy composition retain water, maintain texture, and prevent crystallization. In one embodiment, fructose is also used to increase the sweetness of the gummy composition.

Glucose is an aldose (a sugar with an aldehyde group or CHO) in its straight chain form, but often undergoes an internal cyclization to yield α-D-glucopyranose and β-D-glucopyranose. In gummy composition, glucose may be used to maintain the stability and texture of the gummy and prevent crystallization.

Psicose, Sorbose, and Tagatose are hexoketoses and are C3-C5 epimers of fructose. Psicose is nearly identical to sucrose (table sugar) in sweetness but has nearly zero calories and does not promote tooth decay. Tagatose is nearly a sweet as sucrose yet only has 38% of the caloric value of sucrose and is much more tooth friendly than sucrose. Sorbose is equivalent to sucrose in sweetness. Since these are sugars like fructose, glucose, and sucrose, they behave like sugars without the caloric significance of sucrose and non-cariogenic. In addition, they have advantage over sugar alcohols as their sweetness profile matches that of sucrose, whereas the sugar alcohols can have a metallic taste in addition to their sweetness. The disclosed gummy composition can be made with any of these as a single carbohydrate source, or in combination with each other, or in combination with other sugars or sugar-alcohols.

Trehalose, also known as mycose or tremalose, is a natural alpha-linked disaccharide formed by an α,α-1,1-glucoside bond between two α-glucose units. Trehalose is a non-reducing sugar. Trehalose is reported to have many significant neurological benefits.

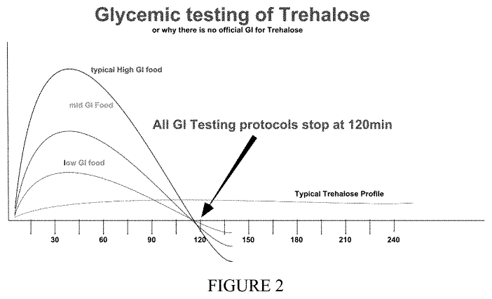

Trehalose is digested in the small intestine by the enzyme trehalase to release two molecules of glucose. The digestion of trehalose does not lead to a spike in glucose levels in the blood (FIG. 2). Rather, the glucose levels slowly rise and are sustained over a longer period of time. Due to the digestion of trehalose in the small intestine rather than the mouth, trehalose has a much lower instance of causing dental decay than most carbohydrates.

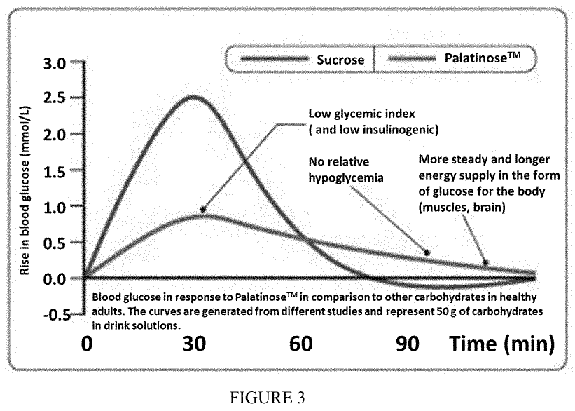

Palatinose (the common or trade name, isomaltulose) is a derivative of sucrose. Palatinose is made by enzymatic rearrangement of the alpha-1,2 bond between the glucose and the fructose molecule to an alpha-1,6 bond. Palatinose is digested by the enzyme sucrase. However, due to the rearrangement of palantinose vs. sucrose, sucrase hydrolysis of palatinose is much slower. Palatinose is slowly metabolized by the body. Like trehalose, palatinose only has a minor influence on the blood glucose level therefore having a low glycemic index (around 30) (FIG. 3). Palatinose also has lower instances of tooth decay.

Sugar Alcohols

Sugar alcohols may include sorbitol, mannitol, erythritol, xylitol, isomalt and maltitol. They are non-cariogenic and non-caloric. Sugar alcohols may be used to mask other flavors. In one embodiment, mannitol may be used to mask bitterness. Mannitol masks bitterness by a mechanism that involves the endothermic nature of mannitol dissolving into water.

Taurine

Taurine, or 2-aminoethanesulfonic acid, may be used to reduce bitterness. In one embodiment, taurine is used to reduce bitterness by 50% when added at a concentration of 300 mM.

Cyclic Sugar

Complexing agents useful in the disclosure may be various cyclic sugars.

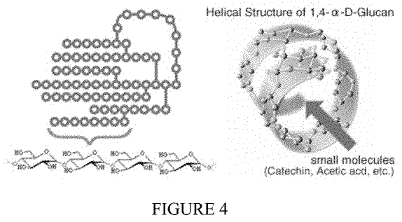

In one embodiment, the cyclic polymers of glucose may be used. The ring structures of the cyclic polymers of glucose may be highly branched. In one embodiment, the cyclic polymers of glucose may be cluster dextrin. A simplified structure of cluster dextrin is shown in FIG. 4.

Cluster dextrin may have a MW from about 1 kDa to about 400 kDa. Cluster dextrin has a ring structure with many branches of long chains of glucose units pendent to the ring. This has the effect of forming a helical structure. The helical structure along with the ring structure of cluster dextrin are both able to chelate small molecules. In one embodiment, the helical structure along with the ring structure of cluster dextrin are used to chelate cannabidiol derivative molecules. The chelation takes place by the hydrophobic groups on the cannabidiol derivative molecules fitting inside the helical structure. In one embodiment, the complexing component comprises cluster dextrin and the semi-solid composition comprises at least 0.1% of cluster dextrin.

In one embodiment, the complexing component comprises cyclodextrin. Cyclodextrins (sometimes called cycloamyloses) are a family of compounds made up of sugar molecules bound together in a ring (cyclic oligosaccharides). Cyclodextrins are composed of 5 or more α-D-glucopyranoside units linked 1->4. Typical cyclodextrins contain a number of glucose monomers ranging from six to eight units in a ring, creating a cone shape. The largest cyclodextrin contains 32 units of 1,4-anhydroglucopyranoside.

Cyclodextrin molecules may have substitution on the ring of α-D-glucopyranoside units. Some examples of moiety substitution on the ring of α-D-glucopyranoside units include hydroxypropyl, methyl, ethyl, acetyl, butyrate, iodo, amino, azido, carboxymethyl and the like. Substitution upon the α-D-glucopyranoside units can modify the interior cavity of the cyclodextrin.

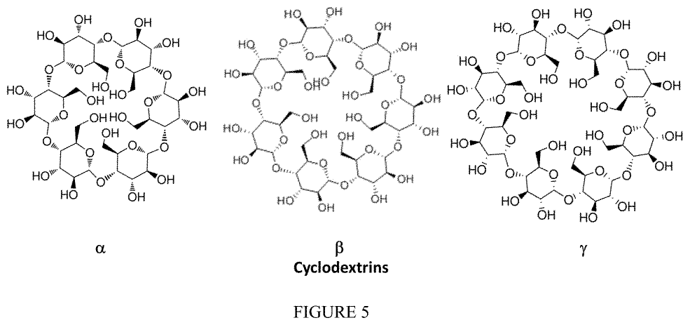

Cyclodextrin may be alpha, beta, or gamma, as shown in FIG. 5. In one embodiment, cyclodextrin may have a MW from about 950 g mol-1 to about 3400 g mol-1. α (alpha)-cyclodextrin is a 6-membered sugar ring molecule. β (beta)-cyclodextrin is a 7-membered sugar ring molecule. γ (gamma)-cyclodextrin is a 8-membered sugar ring molecule. The ring structures form a crown. The interior of the cyclodextrin, be it alpha, beta or gamma, is hydrophobic while the exterior of the cyclodextrin is hydrophilic. The inside of the crown is able to coordinate or complex with hydrophobic molecules.

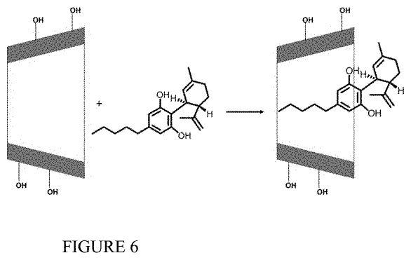

FIG. 6 shows the process of complexing of the CBD molecule. Either the phenyl group or the 4-chlorophenyl group fit inside the ring structure. The formation of the complexing structure is endothermically favorable due to electrostatic interactions of the pi system of the aromatic moiety with the hydroxyl groups. It is these electronic interactions between the hydroxyl moieties of the cyclodextrin and the pi system that gives the favorable heat of formation. The alpha, beta, and gamma cyclodextrins do not form the complex with cannabinoids equally. The novel inclusion complexes of the disclosure can be prepared by dissolving a selected cannabinoid in a selected cyclodextrin. The amount of cannabidiol derivative and cyclodextrin are selected to give desired complexation efficiency, which also depends on the complexation constant between cannabidiol derivative and the cyclodextrin. The complexation constant (K1:1, K1:2) between cannabidiol derivative and cyclodextrins are in a range of 1 M⁻¹ to 100 000 M⁻¹. Beta-CD typically gives the strongest stability constants followed by gamma. In some embodiments, cannabinoids may bind to beta-cyclodextrin with a formation constant up to 20,000 M⁻¹. In one embodiment, CBD may bind to beta-cyclodextrin with a stability constant up to 500000 M⁻¹. In one embodiment, cyclodextrins may enhance cannabinoid permeability through mucosal tissues, allowing the quick absorption and action through the chewing of the semi-solid chewable gel composition.

In one embodiment, the semi-solid chewable gel composition includes alpha-cyclodextrin in an amount not exceeding 3% of the composition weight. In one embodiment, the semi-solid chewable gel composition includes beta-cyclodextrin not exceeding 50 mg/kg.

In one embodiment, the semi-solid composition comprises at least 0.1% of cyclodextrin. In one embodiment, cyclodextrin comprises alpha, beta, gamma-cyclodextrin or a combination thereof. In one embodiment, the complexing component comprises alpha-cyclodextrin, beta-cyclodextrin, gamma-cyclodextrin, or a combination thereof. In one embodiment, the complexing component consists essentially of alpha-cyclodextrin. In one embodiment, the complexing component consists essentially of beta-cyclodextrin. In one embodiment, the complexing component consists essentially of gamma-cyclodextrin. In one embodiment, the semi-solid composition comprises from about 0.05% to about 1% of gamma-cyclodextrin. In one embodiment, the semi-solid composition comprises at least 0.05%, 0.1%, 0.2% or 0.5% of gamma-cyclodextrin.

The coordination or complexing of cannabidiol derivatives by cluster dextrins is reversible allowing the dissociating and releasing of cannabidiols from the formulation after being consumed. Cluster dextrins have a broad range of cyclic ring and helical structures. Statistically some cyclic and helical structures meet the criteria for chelation (complexing) of cannabinoids. The method of chelation of cannabinoids by cluster dextrins is the same electronic interactions that occur as the alpha, beta, and gamma cyclodextrins.

In some embodiments, the semi-solid chewable gel compositions may contain CBD/CBG oil at 3%-20% by weight. The percentages may include both cannabinoids, for example the total weight percentage of CBD/CBG in the composition is 15%, as used in the examples below. CBG may be present at twice to three times the amount of CBD in the same composition. Other ratios of CBG:CBD are contemplated. Other cannabinoids may be present at lower concentration, such as lower than 1% by weight. It is contemplated that CBD/CBG composition at 3-20% by weight percent of the total composition may be used in this treatment method according to embodiments. CBD/CBG concentration in the composition may be more than 10% by weight of the composition.

In some embodiments, cannabinoids may be incorporated into the composition from crystalline and/or powder form. Cannabinoids used in these embodiments may be at a high purity, such as 99%. In some embodiments, cannabinoids concentration in the composition may be at 0.5-20% of the total composition by weight.

Crystalline cannabinoids may be isolated from cannabis extraction. Crystalline cannabinoids may be combined with vegetable oil such as hemp seed oil, coconut oil, palm oil, or a combination thereof.

In some embodiments, the cannabinoids in the formulation may be in nano-encapsulated form. In some embodiment, the size of the encapsulated particles may be between 20 and 40 nm. The cannabinoids in these compositions may also be microencapsulated. Cannabinoids may be sourced naturally or synthetically.

In some embodiments, each gummy piece may contain CBD at 5 mg, 10 mg, 20 mg, 30 mg, 40 mg, 50 mg, 60 mg, 70 mg, 80 mg, 90 mg, 100 mg, 200 mg, 300 mg, 400 mg, 500 mg a piece, or any quantity in between the ranges. Gummy may be consumed by mastication, upon which at least a portion of CBD may be released and absorbed through the oral mucosa.

In some embodiments, different cannabinoids may be included in the gummy formulation provided for treating different conditions. Other cannabinoids may include without limitation CBG, THC, THCV, CBDV, or a combination thereof. Cannabinoids may be present at 0.01 to 10% by total weight of the composition.

The complexing component is capable of interacting with the cannabinoid component through coordinating, chelating, complexing, hydrogen-bonding, dipole-dipole interaction, van-der waals interaction, electrostatic interaction, or a combination thereof. Through complexing with the cannabinoid component, the complexing component acts to reduce the bitterness of the cannabinoid component.

In one embodiment, the complexing component may contain polymeric molecules having a MW of at least 30 kDa. The polymeric molecule may possess tertiary structure capable of complexing with or fitting the cannabinoid component into the structure through hydrogen-bonding, dipole-dipole interaction, van-der waals interaction, or a combination thereof. In one embodiment, the complexing component is configured to complex with cannabinoid.

In one embodiment, the complexing component contains nucleic acid, nucleotide, nucleic acid bases, fruit power, protein, peptide, cluster dextrin, cyclodextrin, polydextrose, polyethylene glycol, fatty acids, waxes, zeolite, chitosan, poly N-acetylglucosamine, or a combination thereof.

In one embodiment, the complexing component comprises DNA, RNA, protein, peptide, resistant starch, porphyrin, polyunsaturated hydrocarbons, polyunsaturated fatty acids, mica, talc, zeolite, silica, cellulose, lignin, plant particles, MOF, calcium carbonate, diatomaceous earth, or a combination thereof.

Nucleic acid may be DNA, RNA, or a combination thereof. Nucleic acid may be extracted from various lifeforms or synthetic. In one embodiment, nucleic acid may have a molecule weight from about 0.2 kDa to about 1000 kDa. Examples of nucleic acids include both DNA and RNA derived from natural sources such as fruit. In one embodiment, the complexing agent comprise an adenosine rich nucleic acid. In one embodiment, the complexing agent comprises thiamin rich nucleic acid. In one embodiment, the complexing component comprises adenosine, cytosine, guanine, thiamine, uracil, or a derivative thereof.

The complexing component may include plant particles or powder. In one embodiment, the plant particles are derived from husk, seed, seed shell, nut, nut shell, fruit, flower, stem, leaf, rice husk, nut shell, woody root, stem or leaves, corn husk, oat husk, grain husk, yeast, mushroom, berry seed, raspberry seed, blackberry seed, blueberry seed, strawberry fruit, chili, pepper, or a combination thereof. In one embodiment, the plant particles comprised defatted berry seed particles. In one embodiment, the plant particles have a particle size from about at least 70 mesh. In one embodiment, the plant particle has a particle size from about 70 to about 200 mesh. In one embodiment, the plant particle has a particle size of not greater than about 200 mesh.

In one embodiment, the plant powder may include fruit powders. Fruit powder may be strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, acai fruit powder, cashew false fruit powder, monk fruit powder, dragon fruit powder, passion fruit powder, coconut powder, guava powder, jujube powder, date powder, cranberry powder or blueberry powder. In one embodiment, the semi-solid composition comprises at least 0.035%, 0.05%, or 0.1%, 0.2%, 0.3% of strawberry powder. In one embodiment, the semi-solid composition comprises at least 0.05%, 0.1%, 0.2%, or 0.3% of comprises orange peel or pulp powder. In one embodiment, the semi-solid composition comprises at least 0.05%, 0.1%, 0.2%, or 0.3% of lemon peel or pulp powder. In one embodiment, the semi-solid composition comprises at least 0.065%, 0.1%, 0.1%, 0.3%, 0.4%, 0.5%, 0.75%, 1%, or 2% of goji berry powder.

In one embodiment, the complexing component comprises a nucleic acid molecule. The complexing agent may include nucleotides or nucleic acid bases. Example nucleic acid bases may include adenine, cytosine, guanine, thymine, and uracil. In one embodiment, the complexing agent comprises adenosine, in which the cannabinoid molecule is capable of pairing through hydrogen bonding, similar to the adenosine and thiamin base pair formation.

In one embodiment, the nucleic acid molecule may be a DNA molecule. The DNA molecule may form a DNA-cannabinoid complex therefore reducing or modulating the bitterness of cannabinoid. In one embodiment, the DNA-cannabinoid complex may have an arrangement in which the cannabinoid molecule is complexed with DNA double helix with an orientation parallel to the bases. In one embodiment, the cannabinoid molecule complexes with DNA double helix through hydrogen-bonding. In one embodiment, the complexing component comprises DNA molecules from plant source.

In one embodiment, the complexing component may include proteins or peptides. Protein or peptide may have a MW from about 0.5 kda to about 1000 kda. In one embodiment, the peptide may be polylysine. In embodiment, the peptide may have MW of not more than 30 kDa. In one embodiment, the peptide may include FVDVT, AGPHGPPGKDGR, D4E1, GLP-1, collagen, or a combination thereof.

Fatty acids may be saturated or unsaturated. Example fatty acids include without limitation coconut oil or fat, palm oil or fat, cocoa butter, shea butter, lard, bacon fat, milk fat, linseed oil, flax seed oil, hemp oil, safflower oil, cotton seed oil, avocado oil, grape seed oil, olive oil and the like.

Waxes may be carnauba wax, bee's wax, paraffin wax, rice bran wax, sugar cane wax, shellac, or resin or any combination.

The semi-solid composition may include at least 0.01% of the complexing component by weight. In one embodiment, the semi-solid composition may include from about 0.5% to about 10.0% the complexing component by weight. In one embodiment, the semi-solid composition may include from about 1% to about 12% of the complexing component by weight.

In one embodiment, the molar ration of complexing component and the cannabinoid component may be from about 1:1 to about 100:1. In one embodiment, the molar ration of complexing component and the caffeinate component is at least 1:1, 2:1, 5:1, 10:1, or 100:1.

The semi-solid composition can contain surprisingly high concentration of cannabinoid without the taste of significant bitterness from cannabinoid. Cannabinoid content may be a total amount of cannabinoid and the cannabinoid content from the plant powder or extract. In one embodiment, the semi-solid composition may contain from about 0.5% to about 10% of cannabinoid. In one embodiment, the semi-solid composition comprises at least 1%, 1.5%, 2%, 2.5%, 3%, 3.5% or 4% cannabinoid by weight. The weight percentage of cannabinoid may be any number in between the ranges.

In one embodiment, the semi-solid composition comprises at least 0.2%, 0.5%, 1%, 1.1%, 1.3%, 1.5%, 2%, 2.5%, 3%, 3.5%, cannabinoid by weight including any numbers in between.

The semi-solid composition may further comprise comprising an herbal composition, an antioxidant composition, a vitamin composition, a mineral composition, an amino acid composition, an active composition, or a combination thereof.

The herbal composition may provide additional bioactive benefit to the gummy composition, may act synergistically with cannabinoid to, for example, enhance the bioactive effect of the composition. In one embodiment, the herbal component comprises gingko, gotu kola, ginseng, ephedra, cocoa (Theobroma cacao), ashwagandha, astragalus, turmeric, ginger, its extract, powder, or derivative thereof.

In one embodiment, the herbal composition may enhance cannabinoid bioactive effect, reduce side effects, or a combination thereof. In one embodiment, the herbal composition comprises gingko biloba, cocoa flavonoids, theobromine, isoflavonoids, flavonoids, quinones, blueberry extract or isolates, vitamin E, curcumin, ginseng, quercetin, melatonin, or isolates, extracts or derivatives thereof.

In one embodiment, the herbal composition may include gingko biloba, turmeric, ginger, astragalus, Prunella vulgaris, Pueraria montana var. lobata, Salvia miltiorrhiza, Coptis chinensis, Eucommia ulmoides Oliver, cranberry, blackberry, elderberry extract, cranberry, blueberry, grapeseed, saffron, Sangre de grado (dragon's blood), Echinacea, Acmella Oleracea, Helichrysum Umbraculigerum, liverwort (Radula Marginata), cacao (Theobroma cacao), black pepper (Piper nigrum), medicinal mushrooms, lavender, peppermint, calendula, creosote, skullcap, comfrey, chamomile, Astragalus, Angelica, eleuthero, reishi, nutgrass, licorice, schisandra, lemon balm, its extract, powder or derivative thereof.

In one embodiment, the herbal composition comprises gingko biloba. In one embodiment, the weight ratio of cannabinoid and gingko biloba is from about 1:100 to about 100:1. In one embodiment, the weight of cannabinoid and gingko biloba ratio is about 10:1. In one embodiment, the weight ratio of cannabinoid and gingko biloba is about 20:1. In one embodiment, the weight ratio of cannabinoid and gingko biloba is about 2:1. In one embodiment, the weight ratio of cannabinoid and gingko biloba is about 1:1.

In one embodiment, the antioxidant composition comprises wherein the antioxidant composition comprises vitamin A, vitamin E, vitamin C, beta-carotene, alpha-carotene, lycopene, lutein, folic acid, gallic acid, resveratrol, quinone, Coenzyme Q10, selenium, selenium yeast, phenolics, polyphenols, anthocyanins, flavonoids, astaxanthin, canthaxanthin, cryptoxanthin, anthracenes, carotenoids, zeaxanthin, curcumin, or derivatives thereof.

In one embodiment, the vitamin composition comprises vitamin A, B, C, D, E, K or a combination thereof. In one embodiment, vitamin B comprises thiamin (B1), riboflavin (B2), niacin or niacinamid (B3), pantothenic acid (B5), pyridoxines (B6), biotin (B7), folate or folic acid (B9), cobalmin (B12), or their derivative thereof.

In one embodiment, the mineral composition comprises salts of calcium, iron, zinc, magnesium, sodium, chloride, potassium, copper, molybdenum, manganese, phosphorus, iodine, nickel, or selenium, or a combination thereof.

In one embodiment, the amino acid composition comprises an essential amino acid or its derivative thereof. In one embodiment, the amino acid composition comprises branch-chain amino acids. In one embodiment, the amino acid composition comprises leucine, isoleucine, valine, their derivative or a combination thereof. In one embodiment, the amino acid composition comprises a stimulant amino acid or its derivative. Example simulant amino acids include tryptophan, aspartate, N-methyl-D-aspartate (NMDA), L-carnitine, L-theanine, glutamate, glutamine, or their derivatives thereof.

In one embodiment, the active composition may include herbal actives, hormones, or bioactive agents. In one embodiment, the active composition includes caffeine. In one embodiment, the bioactive composition include curcumin, gingerol, willow bark extract, salicylic acid, aspirin, or a combination thereof. In one embodiment, the bioactive composition includes melatonin.

In one embodiment, the semi-solid chewable gel composition may further include sweeteners, food acids, flavoring agents, coloring agents, humectants, bulking agents, fatty acids, triglycerides, plasticizers, emulsifiers, thickeners, preservatives, or a mixture thereof.

In one embodiment, the sweetener comprises sucrose, fructose, glucose, erythritol, xylitol, sugar, glucose syrup, corn syrup, high fructose corn syrup, Trulinose®, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenyl alanine 1-methyl ester, salts thereof, licorice or its extracts or isolates, or a mixture thereof.

In one embodiment, the flavoring agent comprises vanilla, chili oil, gingerol, peperine, capsaicin, peppermint oil, spearmint oil, eucalyptus oil, cinnamon oil, grapefruit oil, menthol, mono-menthyl succinate, menthol ethylene glycol carbonate, menthone glycerol ketal, menthyl lactate, (−)-isopulegol, p-menthane-3,8-diols, (−)-monomenthyl glutarate, oil of wintergreen (methylsalicylate), citrus oils, orange oils, fruit essences, rosemary oil, lavender oil, sage oil, rose extra or oil, clary sage oil, thyme oil, sandalwood oil, basil oil, coriander oil, cypress oil, fleabane oil, frankincense oil, geranium oil, fennel oil, oregano oil, Dalmatian sage oil, tarragon oil, cocoa, pineapple flavor, berry flavors or mixtures or derivatives thereof. In one embodiment, the berry flavor comprises flavors, isolates, extracts, or juices of blueberry, raspberry, strawberry, black current, acai berry, bilberry, blackberry, mulberry, boysenberry, cranberry, elderberry, goji berry, gooseberry, huckleberry, or a combination thereof.

The coloring agent may be synthetic or natural. Example natural coloring agents include, without limitation, plant or fruit extract or juice or powder such as, without limitation, beet extract, strawberry extract, carrot extract, spirulina, cochineal, flower extracts or powders such as rose extract or powder, turmeric extract or powder, curcumin extract or powder.

The semi-solid composition may have a pH from slightly basic, neutral or acidic. In one embodiment, the pH of the composition is less than 3. In one embodiment, the pH of the composition is from about 2.7 to about 2.9. In one embodiment, the pH of the composition is form about 2.91 to about 2.99. In one embodiment, the pH of the composition is from about 3.0 to about 3.5. In one embodiment, the pH of the composition is from about 3.51 to about 3.99. In one embodiment, the pH of the composition is from about 4.0 to about 4.5. In one embodiment, the pH of the composition is from about 4.51 to about 4.99. In one embodiment, the pH of the composition is more than 5.0. In one embodiment, the pH of the composition is about 3 to about 5. In one embodiment, the pH of the composition is about 5 to about 7. In one embodiment, the pH of the composition is about 6 to about 8.

In one embodiment, the semi-solid composition comprises at least 0.2%, 0.4%, 0.5%, 1%, 1.3%, 1.5%, 2%, 3% by weight of cannabinoid. In one embodiment, the composition further comprises Ginkgo biloba. The gelling component consists comprises pectin, carrageenan, gelatin, starch, locust bean gum, xanthan gum, gum Arabic, or some combination of the above. In one embodiment, the semi-solid composition may be melon, raspberry, orange, coconut, pineapple, cinnamon, chili pepper, jalapeno pepper, all spice, anise, licorice, rose, peppermint, mint, caramel, salted caramel, pumpkin spice, cinnamon, or gingerbread flavored. In one embodiment, the semi-solid composition is flavored by ground or powdered spice or fruit material. In one embodiment, the semi-solid composition comprises flavors such as liquid flavorings or extracts.

In another aspect, the application provides methods of making the semi-solid cannabinoid compositions disclosed therein. In one embodiment, the method including combining the cannabinoid component with the complex component to provide a first mixture and combining a gelling component with sugar component and optionally buffer salts to provide a second mixture. Mixing the first mixture and the second mixture and optionally other ingredient to provide a combined mixture. Heat the combined mixture to at least brix 78. In one embodiment, heat the combined mixture to a brix from about 78 to about 83, rom about 79 to about 85, from about 80 to about 87, from about 81 to about 90. In one embodiment, heat the combined mixture to a brix less than about 90. Mold the mixture and allow to cool.

The present disclosure is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

### Example: CBG Gummy

Ingredients: Pectin, Sucrose, Sodium Citrate, Glycerol, Mannitol, Fructose, alpha-Cyclodextrin, Corn Syrup, CBG wax (98% CBG), 50% Citric Acid solution.

To a container was added the water which is then heated to 200° F. Glycerol was then added to the water.

To a separate container was added the pectin, 100 grams of sucrose, and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container were added the remainder of the sucrose, cyclodextrin, fructose and mannitol. These components were mixed until homogenous. The sugar mixture was then added to swelled pectin solution above with rapid mixing while maintaining the boil.

To a separate container was added the corn syrup. The corn syrup was brought to a boil and reduced to a Brix 85. The boiling glucose syrup was then slowly added to the pectin solution slowly with rapid mixing. The mixture was brought to a strong boil.

The system was then heated to a Brix of 72. At a Brix of 72 the CBG wax was added with stirring. The system was then heated to Brix 80. The system was cooled to 210-220° F. at which time the cochineal red was added followed by watermelon flavor and then the citric acid solution.

The completed gummy batter was then poured into molds.

### Example. CBDA-Methyl Ester Gummy

Ingredients: Pectin, Sucrose, Sodium Citrate, Glycerol, Mannitol, alpha-Cyclodextrin, Glucose Syrup, CBDA-methyl ester, 50% Citric Acid solution, Turmeric Yellow, Peach Flavor

To a container was added the water which is then heated to 200° F. Glycerol was then added to the water Followed by the cyclodextrin. Once the cyclodextrin was dissolved the CBDA-methyl ester was added.

To a separate container was added the pectin, 100 grams of sucrose, and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container were added the remainder of the sucrose, fructose and mannitol. These components were mixed until homogenous. The sugar mixture was then added to swelled pectin solution above with rapid mixing while maintaining the boil.

To a separate container was added the glucose syrup. The glucose syrup was brought to a boil and reduced to a Brix 85. The boiling glucose syrup was then slowly added to the pectin solution slowly with rapid mixing. The mixture was brought to a strong boil.

The system was then heated to Brix 79. The system was cooled to 210-220° F. at which time the turmeric yellow was added followed by the flavors and then the citric acid solution.

The completed gummy batter was then poured into silicone molds.

### Example. CBN Real Pomegranate

Ingredients: POMX Pomegranate Powder, Pectin, Fructose, Sucrose, Sodium Citrate, Glycerol, alpha-Cyclodextrin, Glucose Syrup, LHO High CBN, 50% Citric Acid solution, Red, Pomegranate Flavor

To a container was added the water which is then heated to 200° F. Glycerol was then added to the water. POMX powder was then added to the water and dissolved.

To a separate container was added the pectin, 50 grams of sucrose, 50 grams fructose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. At a Brix of 75 the CBN was added. The mixture was brought to a strong boil.

The system was then heated to Brix 79. The system was cooled to 210-220° F. at which time the red color was added followed by the flavors and then the citric acid solution.

The completed gummy batter was then poured into molds.

### Example: CBDV Gummy

Ingredients: CBDV Oil (˜30%), Pectin, Fructose, Sucrose, Sodium Citrate Glycerol, alpha-Cyclodextrin, Mannitol, Orange Complex H powder, Glucose Syrup, 50% Citric Acid solution, Spirulina Blue, Blueberry Flavor

To a container was added the water which is then heated to 200° F. Glycerol was then added to the water.

To a separate container was added the pectin, 50 grams of sucrose, 50 grams fructose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose, mannitol, orange complex H powder and cyclodextrin. These components are mixed until homogenous. The sugar mixture was then added to swelled pectin solution above with rapid mixing and the solution maintained at a gentle boil.

To a separate container was added the glucose syrup. The glucose syrup was brought to a boil and reduced to a Brix 85. The boiling glucose syrup was then slowly added to the pectin solution slowly with rapid mixing. The CBDV oil was then added to the mixing solution when a Brix of 75 was reached. The mixture was brought to a strong boil.

The system was then heated to Brix 80. The system was cooled to 230° F. at which time the blue was added followed by the flavors and then the citric acid solution.

The completed gummy batter was then poured into molds.

### Example: CBD Gummy

Ingredients: Pectin, Sucrose, Fructose, Sodium Citrate, Glycerol, Mannitol, alpha-Cyclodextrin, Coconut Oil, Glucose Syrup, RSO CBD Serum 20:1, 50% Citric Acid solution, Pink, Watermelon Flavor

To a container was added the water which is then heated to 200° F. Glycerol was then added to the water.

To a separate container was added the pectin, 100 grams of sucrose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose, fructose, mannitol and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. Coconut oil and RSO CBD Serum 20:1 were added with mixing. Brix was at 70. The mixture was brought to a strong boil.

The system was then heated to Brix 79. The system was cooled to 210-220° F. at which time the color was added followed by the flavors and then the citric acid solution.

The completed gummy batter was then poured into molds.

### Example: CBD with Cacao Gummy

Ingredient: Pectin, Fructose, Sucrose, Sodium Citrate, Glycerol, Cocao Powder, alpha-Cyclodextrin, Glucose Syrup, CBD Genesis DABWAX (less than 0.3% THC), 50% Citric Acid solution.

To a container was added the water which is then heated to 200° F. Glycerol was then added to the water.

To a separate container was added the pectin, 50 grams of sucrose, 50 grams fructose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose, cacao and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. CBD was then added. Brix was at 64. The mixture was brought to a strong boil.

The system was then heated to Brix 79. The system was cooled to 230° F. at which time the citric acid solution was added.

The completed gummy batter was then poured into molds.

### Example: CBD Gummy with Cacao

Ingredients: Pectin, Sucrose, Fructose, Sodium Citrate, Glycerol, Cocao Powder, alpha-Cyclodextrin, Glucose Syrup, RSO CBD Serum 20:1, 50% Citric Acid solution, Orange Oil,

To a container was added the water which is then heated to 200° F. Glycerol was then added to the water.

To a separate container was added the pectin, 100 grams of sucrose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose, fructose, cocao and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. RSO CBD Serum 20:1 was added with mixing. Brix was at 68. The mixture was brought to a strong boil.

The system was then heated to Brix 79. The system was cooled to 210-220° F. at which time the orange oil flavor and citric acid solution were added.

The completed gummy batter was then poured into molds.

### Example: Non-Cariogenic, Diabetic Safe, Sugar Alcohol Free, CBD Gummy

Ingredients: Tagatose, pectin, citric acid, sodium citrate, ginkgo extract, hemp oil, cocoa oil, psicose, cantaloupe powder, cyclodextrin, paprika organ, cantaloupe flavor.

To a container was added the sodium citrate, citric acid and ginkgo powder. To the container was added the water which is then heated to 200° F.

To a separate container was added the pectin, tagatose, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the psicose and cyclodextrin. These components are mixed until homogenous. The psicose and cyclodextrin mixture was then added to 250 g of water and heated on a 255° F. induction plate with stirring. Cacao butter was added when the mixture reached 200° F.

The pectin solution was added to the psicose solution when the psicose solution began to boil with stirring. The hemp oil extract was then then added to the stirring solution.

The system was then heated to Brix 83 at which time the orange oil flavor and citric acid and color were added.

The completed gummy batter was then poured into molds.

### Example: Non-Cariogenic, Diabetic Safe, Sugar Alcohol Free, CBD Gummy

Ingredients: Tagatose, pectin, citric acid, sodium citrate, ginkgo extract, hemp oil, cocoa oil, psicose, cantaloupe powder, cyclodextrin, paprika orange

To a container was added the sodium citrate, citric acid and ginkgo powder. To the container was added the water which is then heated to 200° F.

To a separate container was added the pectin, tagatose, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the psicose and cyclodextrin. These components are mixed until homogenous. The psicose and cyclodextrin mixture was then added to 250 g of water and heated on a 255° F. induction plate with stirring. Cacao butter was added when the mixture reached 200° F.

The pectin solution was added to the psicose solution when the psicose solution began to boil with stirring. The hemp oil extract was then then added to the stirring solution.

The system was then heated to Brix 83 at which time the orange oil flavor and citric acid and color were added.

The completed gummy batter was then poured into molds.

### Example: Non-Cariogenic, Diabetic Safe, Sugar Free, CBD Gummy

Ingredients: Mannitol, pectin, citric acid, sodium citrate, Ginkgo extract, hemp oil extract, cocoa oil, maltitol, cantaloupe powder, cyclodextrin, color, flavor

To a container was added the sodium citrate, citric acid and ginkgo powder. To the container was added the water which is then heated to 200° F.

To a separate container was added the pectin, mannitol, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the maltitol and cyclodextrin. These components are mixed until homogenous. The maltitol and cyclodextrin mixture was then added to 250 g of water and heated on a 255° F. induction plate with stirring. Cacao butter was added when the mixture reached 200° F.

The pectin solution was added to the maltitol solution when the maltitol solution began to boil with stirring. The hemp oil extract was then then added to the stirring solution.

The system was then heated to Brix 83 at which time the orange oil flavor and citric acid and color were added.

The completed gummy batter was then poured into molds.

### Example: Non-Cariogenic, Diabetic Safe, Sugar Free, CBD Gummy with Monk Fruit Extract

Ingredients: Mannitol, pectin, citric acid, sodium citrate, Ginkgo extract, monk fruit extract, hemp extract, cocoa butter, isomalt, cantaloupe powder, cyclodextrin, paprika orange

To a container was added the sodium citrate, citric acid and ginkgo powder. To the container was added the water which is then heated to 200° F.

To a separate container was added the pectin, monk fruit extract, mannitol, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the isomalt and cyclodextrin. These components are mixed until homogenous. The isomalt and cyclodextrin mixture was then added to 250 g of water and heated on a 255° F. induction plate with stirring. Cacao butter was added when the mixture reached 200° F.

The pectin solution was added to the isomalt solution when the isomalt solution began to boil with stirring. The hemp oil extract was then then added to the stirring solution.

The system was then heated to Brix 83 at which time the orange oil flavor and citric acid and color were added.

The completed gummy batter was then poured into molds.

### Example: Non-Cariogenic, Diabetic Safe, Sugar Free, CBD Gummy with Stevia Extract

Ingredients: Mannitol, pectin, citric acid, sodium citrate, ginkgo extract, hemp extract, cocoa butter, isomalt, cantaloupe powder, cyclodextrin

To a container was added the sodium citrate, citric acid and ginkgo powder. To the container was added the water which is then heated to 200° F.

To a separate container was added the pectin, stevia extract, mannitol, cantaloupe powder, and mixed until homogeneous. This mixture was then added to the hot water with rapid mixing.

The water becomes more viscous as the pectin dissolved. The water was brought to a gentle boil and the mixing continued for 3-5 minutes.

To a separate container are added the isomalt and cyclodextrin. These components are mixed until homogenous. The isomalt and cyclodextrin mixture was then added to 250 g of water and heated on a 255° F. induction plate with stirring. Cacao butter was added when the mixture reached 200° F.

The pectin solution was added to the isomalt solution when the isomalt solution began to boil with stirring. The hemp oil extract was then then added to the stirring solution.

The system was then heated to Brix 83 at which time the orange oil flavor and citric acid and color were added.

The completed gummy batter was then poured into molds.

### Example: Non-Cariogenic, Diabetic Safe, Sugar Alcohol Free, CBD Gummy

Ingredients: Tagatose, pectin, glycerol, citric acid, sodium citrate, ginkgo extract, hemp extract, cocoa butter, psicose, strawberry powder, paprika orange

To a container was added the sodium citrate, and ginkgo powder. To the container was added the water which is then heated to 200° F.

To a separate container was added the pectin, tagatose, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the psicose and cyclodextrin. These components are mixed until homogenous. The psicose and cyclodextrin mixture was then added to 250 g of water and heated on a 255° F. induction plate with stirring. Cacao butter was added when the mixture reached 200° F.

The pectin solution was added to the psicose solution when the psicose solution began to boil with stirring. The hemp oil extract was then then added to the stirring solution.

The system was then heated to Brix 83 at which time the orange oil flavor and citric acid and color were added. To the solution was then added a heated 50% solution of citric and/or malic acids to the syrup with mixing.

The completed gummy batter was then poured into molds.

### Example: Non-Cariogenic, Diabetic Safe, Sugar Free, CBD Gummy

Ingredients: Sorbitol, pectin, glycerol, citric acid, sodium citrate, ginkgo extract, hemp oil, cocoa oil, maltitol, cantaloupe powder, cyclodextrin, paprika orange.

To a container was added the sodium citrate, and ginkgo powder. To the container was added the water which is then heated to 200° F.

To a separate container was added the pectin, sorbitol, cantaloupe powder, and mixed until homogeneous. This mixture was then added to the hot water with rapid mixing. The water becomes more viscous as the pectin dissolved. The water was brought to a gentle boil and the mixing continued for 3-5 minutes.

To a separate container are added the maltitol and cyclodextrin. These components are mixed until homogenous. The maltitol and cyclodextrin mixture was then added to 250 g of water and heated on a 255° F. induction plate with stirring. Cacao butter was added when the mixture reached 200° F.

The pectin solution was added to the maltitol solution when the maltitol solution began to boil with stirring. The hemp oil extract was then then added to the stirring solution. The system was then heated to Brix 83 at which time the orange oil flavor and citric acid and color were added. To the solution was then added a heated 50% solution of citric and/or malic acids to the syrup with mixing.

The completed gummy batter was then poured into molds.

### Example: CBD Gummy

Ingredients: Pectin, Sucrose, Sodium Citrate, Glycerol, Mannitol, Fructose, Cyclodextrin, Coconut Oil, Corn Syrup, grams CBD; 50% Citric Acid solution, red, Watermelon Flavor.

To a container was added the water which is then heated to 200° F. Glycerol was then added to the water.

To a separate container was added the pectin, 100 grams of sucrose, and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container were added the remainder of the sucrose, cyclodextrin, fructose and mannitol. These components were mixed until homogenous. The sugar mixture was then added to swelled pectin solution above with rapid mixing while maintaining the boil.

To a separate container was added the corn syrup. The corn syrup was brought to a boil and reduced to a Brix 85. The boiling glucose syrup was then slowly added to the pectin solution slowly with rapid mixing. Coconut oil was added to the mixture with stirring. The mixture was brought to a strong boil.

The system was then heated to a Brix of 74. At a Brix of 74, CBD was added with stirring. The system was then heated to Brix 82. The system was cooled to 210-220° F. at which time the cochineal red was added followed by watermelon flavor and then the citric acid solution.

The completed gummy batter was then poured into molds.

### Example: CBD Gummy

Ingredients: Water, Pectin, Sucrose, Sodium Citrate, Glycerol, Mannitol, Cyclodextrin, Coconut Oil, Glucose Syrup, CBD, 50% Citric Acid solution, Turmeric Yellow, Coconut Flavor, Pineapple Flavor

To a container was added the water which is then heated to 200° F. Glycerol was then added to the water.

To a separate container was added the pectin, 100 grams of sucrose, and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container were added the remainder of the sucrose, cyclodextrin, fructose and mannitol. These components were mixed until homogenous. The sugar mixture was then added to swelled pectin solution above with rapid mixing while maintaining the boil.

To a separate container was added the glucose syrup. The glucose syrup was brought to a boil and reduced to a Brix 85. The boiling glucose syrup was then slowly added to the pectin solution slowly with rapid mixing. Coconut Oil was added with stirring. The mixture was brought to a strong boil.

The system was then heated to a Brix of 75. At a Brix of 75, CBD was added with stirring. The system was then heated to Brix 81. The system was cooled to 210-220° F. at which time the turmeric yellow was added followed by the flavors and then the citric acid solution.

The completed gummy batter was then poured into molds.

### Example: CBD Gummy with Pomegranate Powder

Ingredients: POMX Pomegranate Powder, Pectin, Fructose, Sucrose, Sodium Citrate, Glycerol, Cyclodextrin, Coconut Oil, Glucose Syrup, CBD, 50% Citric Acid solution, Cochineal Red, Pomegranate Flavor

To a container was added the water which is then heated to 200° F. Glycerol was then added to the water. POMX powder was then added to the water and dissolved.

To a separate container was added the pectin, 50 grams of sucrose, 50 grams fructose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. Coconut oil and Blueberry RSO were added with mixing. Brix was at 65. The mixture was brought to a strong boil.

The system was then heated to Brix 81. The system was cooled to 210-220° F. at which time the turmeric yellow was added followed by the flavors and then the citric acid solution.

The completed gummy batter was then poured into molds.

### Example: CBD Gummy

Ingredients: Pectin, Sucrose, Fructose, Sodium Citrate, Glycerol, Mannitol, Cyclodextrin, Coconut Oil, Glucose Syrup, RSO CBD Serum 20:1, 50% Citric Acid solution, Cochineal Pink, Watermelon Flavor

To a container was added the water which is then heated to 200° F. Glycerol was then added to the water.

To a separate container was added the pectin, 100 grams of sucrose and the sodium citrate and mixed until homogeneous. This mixture was then added to the hot water with rapid mixing.

The water becomes more viscous as the pectin dissolved. The water was brought to a gentle boil and the mixing continued for 3-5 minutes.

To a separate container are added the remainder of the sucrose, fructose, mannitol and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. Coconut oil and RSO CBD Serum 20:1 were added with mixing. Brix was at 70. The mixture was brought to a strong boil.

The system was then heated to Brix 79. The system was cooled to 210-220° F. at which time the color was added followed by the flavors and then the citric acid solution.

The completed gummy batter was then poured into molds.

### Example: CBD Gummy with Cocao

Ingredients: Pectin, Fructose, Sucrose, Sodium Citrate, Glycerol, Cocao Powder, Cyclodextrin, Glucose Syrup, CBD, 50% Citric Acid solution.

To a container was added the water which is then heated to 200° F. Glycerol was then added to the water.

To a separate container was added the pectin, 50 grams of sucrose, 50 grams fructose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose. cocao and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. CBD was added with mixing. Brix was at 64. The mixture was brought to a strong boil.

The system was then heated to Brix 80. The system was cooled to 210-220° F. at which time the citric acid solution was added.

The completed gummy batter was then poured into molds.

### Example: CBD Gummy with Cocao

Ingredients: Pectin, Sucrose, Fructose, Sodium Citrate, Glycerol, Cocao Powder, alpha-Cyclodextrin, Glucose Syrup, CBD 20:1, 50% Citric Acid solution

To a container was added the water which is then heated to 200° F. Glycerol was then added to the water.

To a separate container was added the pectin, sucrose and the sodium citrate and mixed until homogeneous. This mixture was then added to the hot water with rapid mixing. The water becomes more viscous as the pectin dissolved. The water was brought to a gentle boil and the mixing continued for 3-5 minutes.

To a separate container are added the remainder of the sucrose, fructose, cocoa and cyclodextrin. These components are mixed until homogenous. The sugar mixture was then added to swelled pectin solution above with rapid mixing and maintain the boil.

To a separate container was added the glucose syrup. The glucose syrup was brought to a boil and reduced to a Brix 85. The boiling glucose syrup was then slowly added to the pectin solution slowly with rapid mixing. CBD was added with mixing. Brix was at 68. The mixture was brought to a strong boil.

The system was then heated to Brix 79. The system was cooled to 210-220° F., at which time the citric acid solution was added.

The completed gummy batter was then poured into molds.

While the disclosure has been particularly shown and described as referenced to the embodiments thereof, those skilled in the art will understand that the foregoing and other changes in form and detail may be made therein without departing from the spirit and scope. All references cited or referred to in this disclosure are hereby incorporated by reference in their entireties.

What is claimed is:

1. A semi-solid chewable gel composition, comprising,
a gelling component in a sufficient amount to provide a cohesive gelled product,
a cannabinoid component comprising cannabidiol, and
a complexing component comprising nucleic acid, nucleotide, nucleic acid base, plant powder, protein, peptide, cluster dextrin, cyclodextrin, polydextrose, polyethylene glycol, fatty acids, waxes, zeolite, chitosan, poly N-acetyglucosamine, N-acetylglucosamine, or a combination thereof, wherein the semi-solid chewable gel composition comprises at least 0.5% the complexing component by weight, and wherein the complexing component is configured to reduce the bitterness of the cannabinoid component by complexing with the cannabinoid component.

2. The semi-solid chewable gel composition of claim 1, wherein the cannabinoid component comprises cannabidiol (CBD), delta9-tetrahydrocannabinol (THC), cannabichromene (CBC), cannabigerol (CBG), cannabinol (CBN), cannabidivarin (CBDV), a cannabis plant extract or isolate, a hemp extract or isolates, a marijuana extract or isolate, or a combination thereof.

3. The semi-solid chewable gel composition of claim 1, wherein the cannabinoid component comprises substantially cannabidiol.

4. The semi-solid chewable gel composition of claim 1, wherein the cannabinoid component comprises less than 0.3% of THC.

5. The semi-solid chewable gel composition of claim 1, wherein the semi-solid chewable gel composition comprises at least 3% the complexing component by weight.

6. The semi-solid chewable gel composition of claim 1, wherein the complexing component comprises cyclodextrin and wherein the semi-solid chewable gel composition comprises at least 0.1% of cyclodextrin.

7. The semi-solid chewable gel composition of claim 1, wherein the complexing component comprises a plant powder and wherein the semi-solid chewable gel composition comprises at least 0.5% of the plant powder, wherein the plant powder comprises strawberry powder, cantaloupe powder, pineapple powder, raspberry powder, blueberry powder, acai berry powder, goji berry powder, citrus fruit powder, cranberry powder, coffee cherry powder, cherry powder, apple powder, peach powder, pear powder, banana powder, plum powder, dragon fruit powder, passion fruit powder, starfruit powder, jackfruit powder, jujube powder, cranberry powder, date powder, pomegranate fruit powder, mango powder, papaya powder, or a combination thereof.

8. The semi-solid chewable gel composition of claim 1, wherein the gelling component comprises gelatin, starch, pectin, gellan gum, gum Arabic, carrageenans, guar, agar, alginate, locust bean gum, xanthan, or a combination or derivatives thereof.

9. The semi-solid chewable gel composition of claim 1, wherein the gelling component consists essentially of pectin having a methoxy content of not less than 40%.

10. The semi-solid chewable gel composition of claim 1, wherein the gelling component consists essentially of pectin having an amid content of not less than 12%.

11. The semi-solid chewable gel composition of claim 1, further comprising a binding component, wherein the binding component comprises sucrose, glucose, fructose, palatinose, trehalose, psicose, tagatose, sorbose, maltodextrin, resistant maltodextrin, a sugar alcohol, or a combination thereof.

12. The semi-solid chewable gel composition of claim 11, wherein the binding component comprises essentially trehalose, palatinose, psicose, tagatose, sorbose, maltodextrin, resistant maltodextrin, or a combination thereof.

13. The semi-solid chewable gel composition of claim 11, wherein the binding component comprises essentially sugar alcohols.

14. The semi-solid chewable gel composition of claim 11, wherein the binding component comprises sorbitol, xylitol, maltitol, isomalt, lactitol, mannitol, erythritol, hydrogenated starch hydrolysates, or a combination thereof.

15. The semi-solid chewable gel composition of claim 1, wherein the semi-solid chewable gel composition comprises at least 0.5% cannabidiol by weight.

16. The semi-solid chewable gel composition of claim 1, further comprising an antioxidant composition, a vitamin composition, a mineral composition, an amino acid composition, an herb composition, a prebiotic composition, a probiotic composition, an active composition, or a combination thereof.

17. The semi-solid chewable gel composition of claim 16, wherein the herbal composition comprises gingko biloba, turmeric, ginger, willow bark, astragalus, angelica, Prunella vulgaris, Pueraria montana var. lobata, Salvia miltiorrhiza, Coptis chinensis, Eucommia ulmoides Oliver, elderberry extract, grapeseed, saffron, Sangre de grado (dragon's blood), green tea, ginseng, Ashwagandha, chamomile, lavender, passion flower, Magnolia bark, Valerian root, lemon balm, kava, St. John's wort, Rhodiola (Rhodiola Rosea), maca, rosemary, thyme, peony, Albizia julibrissin, icariin, Epimedium, Perilla frutescens, its extract, powder or derivative thereof.

18. The semi-solid chewable gel composition of claim 16, wherein the active composition comprises glucosamine, chondroitin, melatonin, caffeine, theobromine, gamma-aminobutyric acid, L-theanine, curcumin, gingerol, or a combination thereof.

19. The semi-solid chewable gel composition of claim 1, having a glycemic index of not more than 10.

20. The semi-solid chewable gel composition of claim 1, having a glycemic index of more than 80.
